(19) United States
(12) Patent Application Publication
(10) Pub. No.: US 2014/0193500 A1
(43) Pub. Date: Jul. 10, 2014

(54) DRY SHAMPOO COMPRISING UREA-FORMALDEHYDE

(71) Applicants: Phillip Loraine Cotrell, Salisbury, NC (US); Corinne Anne-Marie Snider, Massena, NY (US); Philip Arie Matena, Runsom, NJ (US)

(72) Inventors: Phillip Loraine Cotrell, Salisbury, NC (US); Corinne Anne-Marie Snider, Massena, NY (US); Philip Arie Matena, Runsom, NJ (US)

(21) Appl. No.: 14/110,988

(22) PCT Filed: Apr. 3, 2012
(86) PCT No.: PCT/GB2012/050754

§ 371 Date: Dec. 18, 2013

Related U.S. Application Data
(60) Provisional application No. 61/476,502, filed on Apr. 18, 2011.

(51) Int. Cl. A61K 8/84; A61Q 5/06; A61Q 5/12; A61Q 5/02; A61Q 19/00; A61Q 5/00

(52) U.S. Cl. CPC A61K 8/84; A61Q 19/00; A61Q 5/00; A61Q 5/12; A61Q 5/02; A61Q 5/008; A61Q 5/06
USPC 424/489; 424/70.11; 424/78.03

(57) ABSTRACT

A method of treating hair and/or skin of a human or animal, the method comprising applying to the hair and/or skin a composition comprising particles of a urea formaldehyde polymer wherein the particles have an average size of less then 300 microns; an oil absorption value of greater than 40 g/100 g; and a bulk density of greater than 0.2 gcm⁻³.

The present invention relates to methods and compositions for application of the skin and/or hair. In particular the invention relates to the application of compositions to the skin and/or the hair in order to absorb excess bodily fluids and remove other contaminants, residue or dirt therefrom.

Glands in the skin of mammals secrete an oily or waxy matter called sebum which lubricates the skin and hair. The material is unharmful and is believed to have benefits for the skin and hair. However excessive sebum is generally undesirable and many people are keen to remove this. The skin also produces sweat which can again build up on the skin and it is often desired to remove this. The skin and hair may also pick up other soils, for example environmental pollutants, debris, dirt and odours and it may be desirable to remove these.

Very many methods are known for removing sebum, sweat and other bodily fluids from the hair and/or skin but each has its disadvantages. The present invention seeks to provide an improved method by which contaminants and/or excess bodily fluids can be removed from the surface of the skin and/or hair.

According to a first aspect of the present invention there is provided a method of treating the hair and/or skin of a human or animal, the method comprising applying to the hair and/or skin a composition comprising particles of a urea formaldehyde polymer wherein the particles have an average size of less then 300 microns; an oil absorption value of greater than 40 g/100 g; and a bulk density of greater than 0.2 gcm⁻³.

According to a second aspect of the present invention there is provided a personal care composition comprising particles of a urea formaldehyde polymer having an average particle size of less than 300 microns; an oil absorption value of greater than 40 g/100 g; and a bulk density of greater than 0.2 gcm⁻³.

Preferably the method of the first aspect involves applying to the skin and/or hair a composition of the second aspect.

Preferred features of the method and composition of the present invention will now be defined.

The present invention involves a composition comprising particles of a polymer of urea and formaldehyde. This may be referred to herein as a “UF polymer”. This is a polymer of urea and formaldehyde and in preferred embodiments substantially all of the monomers used to prepare the polymer are urea or formaldehyde. The polymers may comprise low levels of other monomers. However in preferred embodiments at least 80% of the monomers used to prepare the UF polymer are urea or formaldehyde. Preferably at least 90%, more preferably at least 99%, for example at least 99.9%.

Preferably the molar ratio of urea monomers to formaldehyde monomers is from 3:1 to 1:3, suitably from 2:1 to 1:2, preferably from 1.5:1 to 1:1.5, more preferably from 1.3:1.1 to 1:1.3, most preferably from 1.2:1 to 1:1.2, for example from 1:1 to 1:1.2.

The polymer may be prepared by any suitable means. Such means will be known to the person skilled in the art. A suitable method is selected to ensure that the polymer produced has the specified properties (such as particle size, bulk density, oil absorption value). A suitable method is described in example 1.

Preferably the UF polymer is not present as a foam. Preferably it is not formed from a foam.

Preferably the composition comprises the urea formaldehyde polymer in an amount of at least 1 wt %, preferably at least 2.5 wt %, preferably at least 5 wt %, suitably at least 10 wt %, preferably at least 15 wt %, more preferably at least 20 wt %, preferably at least 25 wt %, preferably at least 30 wt %, more preferably at least 35 wt %.

Suitably the UF polymer is present in the composition in an amount of up to 100 wt %, preferably up to 95 wt %, suitably up to 90 wt %, preferably up to 85 wt %, for example up to 80 wt %, suitably up to 75 wt %, preferably up to 70 wt %, preferably up to 65 wt % and more preferably up to 60 wt %, for example up to 55 wt %.

Preferably the particles of the urea formaldehyde polymer have an average particle size of at least 10 microns, suitably at least 20 microns, preferably at least 30 microns, more preferably at least 35 microns, for example at least 40 microns. In especially preferred embodiments the UF polymer has an average particle size of at least 50 microns.

Suitably the UF polymer has an average particle size of less than 300 microns, preferably less than 200 microns, suitably less than 120 microns, for example less than 110 microns or less than 105 microns.

Preferably the UF polymer has an average particle size of less than 100 microns, preferably less than 95 microns, more preferably less than 90 microns.

Preferably the average particle size of the UF polymer is from 50 to 80 microns, preferably from 55 to 75, for example from 60 to 70 microns.

Particle sizes defined herein are determined by laser light scattering. Such methods will be known to the person skilled in the art. One suitable technique is described in example 7.

Preferably 90% of the particles of the UF polymer have a particle size of less than 105 microns. Preferably 95% of the particles have a particle size of less than 165 microns. Preferably 97% of the particles have a size of less than 165 microns, preferably less than 140 microns, more preferably less than 120 microns.

Preferably at least 90% of the particles have a particle size of greater than 10 microns, preferably greater than 97%. Preferably 90% of the particles have a particle size of greater than 23 microns.

The particles of the urea formaldehyde polymer used in the present invention are generally porous.

Preferably the UF polymer used in the present invention has an oil absorption value of at least 40 g/100, preferably at least 60 g/100 g, more preferably at least 75 g/100 g and most preferably at least 90 g/100 g as measured by standard method ASTM D281.

Preferably the UF polymer has an oil absorption value of greater than 110 g/100 g, preferably greater than 130 g/100 g.

Preferably the particles of the UF polymer have a compact and uniform structure.

Preferably the UF polymer has a bulk density of at least 0.2 gcm⁻³, preferably at least 0.3 gcm⁻³. The bulk density may be up to 1.0 gcm⁻³, for example up to 0.7 gcm⁻³. The bulk density may suitably be determined by conventional techniques such as will be known to the person skilled in the art. Such techniques involve weighing a quantity of particles which fill a calibrated container.

For the avoidance doubt the above definitions of particle size, oil absorption value and bulk density refer to dried particles of the UF polymer themselves, before they are incorporated in to a composition.

The composition of the present invention is preferably in the form of a solid. Preferably it is in the form of a particulate material, especially a fine particulate material such as a powder.

Although the composition of the present invention is preferably a solid composition and is preferably used in the particulate form, it preferably includes one or more ingredients which are provided in liquid form. Preferably the weight ratio of total weight of liquid ingredients to UF polymer particles is from 10:1 to 1:10, for example from 8:1 to 1:8, suitably from 6:1 to 1:6, preferably from 4:1 to 1:4, for example from 1:2 to 2:1.

The composition may comprise one or more further ingredients (in addition to the UF polymer) which are usually provided in solid form. Suitably the weight ratio of the total solid ingredients to total liquid ingredients is from 15:1 to 1:10, preferably from 12:1 to 1:8, suitably from 10:1 to 1:6, preferably from 8:1 to 1:4, suitably from 6:1 to 1:3, preferably from 5:1 to 1:2, for example from 3:1 to 1:1.2.

Suitable components which may be provided in liquid form include water, organic solvents, surfactants and mixtures thereof. Although some surfactants suitable for use in the present invention may be supplied as a paste or solid, they are referred to herein as a liquid ingredient or component since they dissolve in the other liquids present.

When mixed together the particles of the solid components may be coated by the liquid components. In preferred embodiments the UF polymer is porous and at least some of the liquid components are absorbed into the particles of the polymer.

Although the composition of the present invention may be prepared from both solid and liquid ingredients the bulk composition itself is preferably in the form of a particulate material especially a powder.

Preferably the composition comprises water. Preferably the weight ratio of water to all other liquid components is at least 0.5:1, preferably at least 1:1, for example at least 2:1.

Preferably the composition comprises at least 1 wt % water, preferably at least 2.5 wt %, more preferably at least 5 wt %, suitably at least 7.5 wt %, preferably at least 10 wt %, more preferably at least 12.5 wt %, preferably at least 15 wt %, suitably at least 20 wt %, suitably at least 25 wt %, for example at least 27.5 wt % or at least 30 wt %.

The composition may comprise up to 70 wt % water, for example up to 65 wt %, preferably up to 60 wt %, preferably up to 55 wt %, suitably up to 50 wt %, preferably up to 45 wt %, for example up to 40 wt %.

Suitable organic solvents which may be used in the compositions of the present invention include hydrocarbons, alcohols, glycols, polyalkylene glycols, esters, glycerine and mineral oil.

Suitably the organic solvent may be present in the composition in an amount of at least 0.1 wt %, suitably at least 0.25 wt %, preferably at least 0.5 wt %, preferably at least 1 wt % for example at least 2 wt %, at least 3 wt %, at least 4 wt % or at least 5 wt %. An organic solvent may be present in the composition in an amount of up to 50 wt %, for example up to 45 wt %, up to 40 wt %, up to 35 wt % or up to 30 wt %.

In some preferred embodiments the composition further comprises a surfactant. As mentioned above the surfactant may be one which is normally solid or liquid under standard conditions but in the compositions of the invention the surfactant is referred to as part of the liquid components.

Suitable surfactants for use herein include anionic surfactants, cationic surfactants, amphoteric surfactants, non-ionic surfactants, and mixtures thereof.

Suitable anionic surfactants include but are not limited to alkyl sulfates, alkyl ether sulfates, alkyl ether sulfosuccinates, alkyl sulfosuccinates, sodium acyl sarcosinates, acyl isethionates, acyl methyl isethionates, alkyl benzene sulfonates, alkyl ether carboxylates, alpha olefin sulfonates, sodium methyl acyl taurates, fatty acid soaps and sulfoacetates.

Suitable cationic surfactants include but are not limited to stearalkonium chloride, cetrimonium chloride, dialkyl dimethyl ammonium chloride, alkyl dimethylamine salts, and alkyl and dialkyl ammonium methylsulfate salts.

Suitable amphoteric surfactants include but are not limited to alkylamphoacetates, dialkylamphodiacetates, amine oxides, alkylamidohydroxypropyl sultaines, alkyl betaines, alkylamidopropyl betaines and alkyl and dialkyl propionates.

Suitable non-ionic surfactants include but are not limited to alkyl polyglucosides, alcohol ethoxylates, benzoate esters, fatty acid esters, alkyl ether esters, alkyl ester ethoxylates (PEG esters), EO/PO block polymers, EO/PO random polymers and fatty alcohols.

Surfactants are preferably present in the composition in an amount of from 0.001 to 10 wt %, suitably from 0.01 to 8 wt %, preferably from 0.05 to 6 wt %, for example from 0.1 to 5 wt %, typically from 0.25 to 3 wt %.

In some embodiments a mixture of two or more surfactants and/or two or more organic solvents may be present. In such embodiments the above amounts relate to all such components present in the composition.

The composition of the present invention may comprise a mixture of liquid ingredients. This mixture of liquid ingredients when combined preferably has a surface tension of less than 40 dynes/cm. Surface tension may be measured by any standard method, for example the DeNoOy Ring method.

In some preferred embodiments the composition further comprises one or more solid adjuvant components. Preferred solid adjuvants are inorganic salts. Suitable inorganic adjuvant salts include alkali and alkaline earth metal salts of sulfates, chlorides, carbonates, bicarbonates, borates, citrates, phosphates, nitrates, metasilicates and mixtures thereof. Especially preferred inorganic salts are alkaline earth metal salts, especially salts of magnesium and most preferably calcium. One especially preferred solid adjuvant salt for use herein is calcium carbonate.

The solid adjuvant salt preferably has an average particle size of from 45 to 600 microns. It may include water of hydration but is otherwise supplied as a dry ingredient. Water of hydration is defined herein to include all water that cannot be driven off by heating a 1 to 1.5 g sample at 10° C. for 2 hours.

Preferably the inorganic solid adjuvant salt is present in an amount of at least 0.1 wt %, preferably at least 0.5 wt %, suitably at least 0.1 wt %, for example at least 2.5 wt %, preferably at least 5 wt %, preferably at least 10 wt %, more preferably at least 12 wt %, suitably at least 15 wt %, more preferably at least 18 wt %.

The solid adjuvant salt maybe present in an amount of up to 70 wt %, preferably up to 60 wt %, suitably up to 55 wt %, preferably up to 50 wt %, more preferably up to 40 wt %, suitably up to 35 wt %, for example up to 30 wt % or up to 25 wt %.

When a mixture of adjuvant salts is present the above amounts refers to the total of all such salts present in the composition.

The composition may further comprise one or more optional further ingredients. These may be provided as liquid and/or as solid components. Suitable further ingredients include preservatives, dyes, fragrances, anti-caking agents, hair conditioning compounds, soft hold polymers, film-forming agents, thickeners, vitamins, proteins, UV filters, sunscreens, anti itch additives, anti fungal additives and anti lice additives.

Suitable preservatives for use herein include methylchloroisothiazolinone, methylisothiazolinone and combinations thereof (for example as sold under the trade mark Kathon CG); potassium sorbate; DMDM hydantoin; methyl, propyl and ethyl parabens; benzoic acid and/or its sodium salt, phenoxyethanol and those sold under the trade marks Quaternium 15 or Dowicil 200. Preservatives are preferably included in the composition in an amount of less than 2 wt %. Mixtures of preservatives may be used.

Suitable dyes or colourants may be included to mask the effect of any residual product left on the skin and or hair after treatment. Suitable dyes for use herein include synthetic dye compounds and mineral compounds such as iron oxide.

Suitable fragrances for use herein will be known to the person skilled in the art and are present in an amount of less than 1 wt %.

Suitable hair conditioning compounds for use herein include guar compounds including cationic polymers and guar gum; polycationic compounds designated as polyquaternium 4, 6, 7, 10 or 22; and silicones including phenyl trimethicones, dimethicones, cyclomethicones, dimethicone copolyols and amino silicones.

Suitable soft hold polymers for use herein include polyquaternium 11, PVP, VP copolymers, acrylate copolymers and derivatives thereof.

Suitable film-forming agents for use herein include waxes such as candelilla wax.

Suitable thickeners for use herein include sodium polyacrylates, acrylate copolymers, carbomers, acrylates/C10-30 alkyl acrylate crosspolymers, polyacrylate-1 cross polymers, gums such as xanthan gum and other carbohydrates and derivatives thereof.

The method of the present invention involves treating the hair and/or the skin with a composition comprising a UF polymer and one or more further ingredients as defined above. Suitably the method involves applying the composition to the skin and/or hair; leaving the composition on the skin and/or hair; and optionally removing some or all of the composition from the skin and/or hair.

The composition of the invention suitably absorbs bodily fluids, for example sweat and/or sebum from the hair and/or skin. The composition may also be used to remove other contaminants from the skin and/or hair, for example environmental pollutants, dirt, debris, odour-causing particles and product residues.

In some embodiments the composition may be applied to the skin and/or hair and left a short period before removing, suitably along with absorbed bodily fluid. Such a short period may be from 0.1 to 30 minutes, for example from 0.5 to 20 minutes, suitably from 1 to 15 minutes or from 1.5 to 10 minutes.

In some embodiments the composition may be applied to the skin and/or hair and left to absorb bodily fluid over a longer period, for example at least 0.5 hours, at least 1 hour, at least 2 hours or at least 4 hours.

Preferably the composition is applied to the skin and/or hair in particulate form, for example as a powder. It may be applied by any suitable means. For example it may be applied by shaking, spraying, rolling or dusting from a suitable container. Alternatively and/or additionally it may be applied using a suitable applicator, for example a brush or pad.

The composition may be removed from the skin and/or hair by any suitable means. Preferably most or substantially all of the composition is removed, for example at least 70%, at least 80% or at least 90%.

The composition may be removed for example by shaking, brushing, wiping or washing away with water.

The personal care composition of the present invention may be a powdered cleansing composition for example a hair cleansing composition or a skin cleansing composition. The composition may be a cosmetic composition for application to the skin or hair, for example loose, pressed or poured powder compositions for application to the face or body.

The method of the present invention may be used to treat humans or animals. For the avoidance of doubt the term hair when used in the context of animals includes fur.

In some preferred embodiments the composition is a hair treatment composition, preferably for treating human hair. Preferably the composition is used to treat hair growing on a living animal, especially a human. Preferably the composition is used to treat hair growing on the head.

In some embodiments the composition may be a dry shampoo composition. This is suitably applied to the hair, left for a period (typically 0.1 to 20 minutes) to absorb bodily fluids (for example sebum and/or sweat) and then removed from the hair. Preferably it is removed as a powder by brushing the hair but may alternatively be removed by shaking the head or by washing from the hair with water.

In some embodiments the composition is applied to the hair and then removed after a short time period.

In some alternative embodiments the composition may be applied to the hair and left on the hair.

When applied to the hair, the method of the present invention may be used for conditioning, to control static fly away hair, to improve shine, to remove oil from the hair and/or to remove odours from the hair, or to improve dry controllability.

Effects such as conditioning, shine, odour and controllability may be subjective and may be measured by, among others, consumer surveys, panel evaluations etc.

In some embodiments the composition of the present invention may be applied to the skin. It may be left on the skin for a short period of time and then removed by wiping or rinsing with water. Alternatively it may be left on the skin for longer periods.

Compositions applied to the skin may include body cleansing compositions, facial cleansing compositions, compositions for oil removal and underarm deodorants.

The invention will now be further defined with reference to the following non limiting examples.

### EXAMPLE 1

Two dry shampoo compositions were prepared having the following ingredients:

| | Composition A (wt %) | Composition B (wt %) |
|---|---|---|
| Dried urea-formaldehyde | 44 | 44 |
| polymer | | |
| Calcium carbonate | 21 | 21 |
| Non-ionic surfactant | 0.6 | 0.6 |
| Kathon CG | 0.1 | 0.1 |
| (preservative) | | |
| Potassium sorbate | 1 | 1 |
| (preservative) | | |
| Fragrance | 0.03 | 0.2 |
| Water | To 100% | To 100% |

The urea-formaldehyde polymer had an average particle size of 60 to 70 microns with 97% of particles having a diameter of less than 120 microns and less than 10% of particles having a size of less than 23 microns. The material had a bulk density of 0.45 gcm⁻³ and the oil absorption value of the polymer as determined by ASTM D281 was 110 g/100 g. Before taking these measurements the polymer was dried overnight at 110° Celsius and then allowed to cool.

The non-ionic surfactant was an ethylene oxide/propylene oxide block copolymer (from BASF). Kathon CG is a mixed isothiazolinone preservative available from Dow Chemicals.

### EXAMPLE 2

Clean virgin brown hair tresses were selected with 20 cm length, 1.5 cm width and 4-5 g weight. The hair samples were prepared by immersing the tress in 200 ml isopropyl alcohol for 10 minutes. The hair was then soaked in 1.6% sodium lauryl ether sulphate (SLES) solution at 50° C. for 30 minutes. Each tress was rinsed with tap water, washed with 1 ml of 16% SLES for 1 minute and rinsed for 30 seconds. The rinse procedure was repeated a second time. The hair tresses were then air-dried overnight and combed with a standard brush two times.

### EXAMPLE 3

Five substantially identical clean tresses prepared in example 2 were selected each weighing approximately 4.5 g and designated A to E. Tress A was used as a control. The other 4 tresses were treated with jojoba oil as it has sebum-like properties. To each hair tress was uniformly applied approximately 500 microlitres (11% of the hair weight in each case) of jojoba oil. The jojoba oil was spread uniformly on each tress by gentle kneading, rubbing and massage for 1 minute using fingers with latex gloves. The 4 tresses were hung up and left to air-dry overnight. Tress B was left treated with jojoba oil. Tress C was cleaned with dry shampoo composition A of example 1. Tress D was cleaned using a commercially available dry shampoo in a non-aerosol (powder) form. To each of tresses C and D, 3.0 g of powder was added and combed and massaged with a mini-brush before being fluffed with fingers. The tresses were then left for a few minutes before being brushed with a standard brush six times. Tress E was cleaned with a “wet” commercial shampoo by applying 0.5 g shampoo to the tress.

It was lathered with water for 1 min, left for 30 s and rinsed for 30 s and left to air dry overnight and brushed two times. Following the tests, tresses C and E had an appearance similar to that of the control sample A, whereas on tress D a white residue was observed.

### EXAMPLE 4

Two substantially identical clean tresses prepared in example 2 were selected and a commercial baby powder based on corn starch was applied to one of the hair tresses so the hair tress appeared white. Any excess powder was gently removed by shaking and combing the tress for 1 min using fingers with latex glove. 3.0 g of composition A of example 1 was then applied evenly through the tress with a mini brush. The hair was then fluffed with fingers and left a few minutes. The tress was then combed with a standard brush 6 times or until the hair looked like the other untouched or control hair tress.

It was observed that the baby powder was removed leaving an appearance which was similar to the control. It was also observed that fragrance resulting from the baby powder was removed and left the hair with a neutral smell. This illustrates that the composition A of example 1 is suitable for removing solid residues and odours from the hair.

### EXAMPLE 5

Two substantially identical clean tresses prepared in example 2 were selected and a commercial baby powder based on corn starch was applied to one of the hair tresses so the hair tress appeared white. Any excess powder was gently removed by shaking and combing the tress for 1 min using fingers with latex glove. The hair was then blown with hot air for 15 minutes. At the end of this period, it was observed that the hair tress was charged with static and had a severe static flyaway effect. 3.0 g of the composition A of example 1 was then applied evenly through the tress with a mini brush. The hair was then fluffed with fingers and left a few minutes. The tress was then combed with a standard brush 6 times or until the hair looked like the other untouched or control hair tress.

After the treatment with composition A of example 1, the hair tress not only appeared clean, but the hair no longer showed the effects of static and appeared similar to the control.

### EXAMPLE 6

The dry shampoo composition B of example 1 was evaluated by a panel of 32 people who used the product over a period of two weeks. At the end of each use, the panellists completed a questionnaire and the results below show the percentage of occasions on which the panellists agreed with each of the following statements.

| Product Performance Attributes | % Agree |
|---|---|
| Product application is convenient | 70.5 |
| Product brushes out easily | 84.6 |
| Hair looks fresh and feels better | 96.8 |
| Hair has a clean appearance | 98.1 |
| Hair is conditioned | 83.3 |
| Hair is soft and manageable | 85.9 |
| Hair is fresh smelling | 98.7 |
| Maintains hair style | 76.9 |
| Works well | 90.4 |
| Generally, I like the product performance | 82.7 |

### EXAMPLE 7

A urea-formaldehyde polymer used in the present invention may be prepared as follows: 33 Parts formaldehyde (1.1 molar equivalents) and 60 parts urea (1 molar equivalent) were diluted with 300 parts water at ambient temperature. 1 part of hydrochloric acid (37% solution) was added and the mixture agitated for 2 hours. The solid product was collected by filtration and washed on the filter with water until the wash water was non-acidic, before drying in a vacuum oven at 120° C. to 125° C.

### EXAMPLE 8

The particle size of the urea-formaldehyde polymers used in the present invention may be measured using a MicoTrac X-100 Particle Analyser according to the following method.

Powder samples are first dispersed in a liquid then are circulated through an optical cell. Particles in the liquid scatter the laser light, which passes through the optical cell. A photo diode array behind the optical cell detects the scattering pattern created by the particles in the liquid. The scattering pattern is sent to the computer, which then calculates the particle size distribution of the powder.

### Apparatus

1. MicroTrac X-100 system

2. Water reservoir (5 gal capacity)

3. Variable speed stirrer with marine impeller

4. Ultrasonic bath with timer

5. 150 ml beaker

6. Micro spatula (2″× 5/16″ blade)

7. 5 ml disposable pipette

### Procedure

Add 80-100 ml of surfactant to a 150 ml beaker (see surfactant recipe below). Surfactant recipe:

3.2 liters water

55 g Triton H-66

5 g Surfadone LP-100

15 g Ammonium Hydroxide (aq).
Place the 150 ml beaker of surfactant under the stirrer.
Turn the stirrer on then slowly add four micro spatulas of UF polymer powder to the beaker.
Transfer the 150 ml beaker to the ultrasonic bath. Sonicate for 1-2 minutes to disperse any lumps of powder.
Remove the 150 ml beaker from the ultrasonic bath. Place the beaker under the stirrer and turn it on.
Wait for the sample loading screen to indicate “Add sample” then transfer a 5 ml aliquot of liquid from the 150 ml beaker to the Automated Small Volume Recirculator. Wait 10-15 seconds for the system to stabilize then add more sample if required.

### Other Parameters

Particle Refractive Index: 1.58

Fluid Refractive Index: 1.33

Flow Rate in the Automated Small Volume Recirculator: 40 ml/sec

Ultrasonic Power: 30 watts

Ultrasonic time: 60 sec

### EXAMPLE 9

The bulk density of the urea-formaldehyde polymers used in the present invention may be measured as follows:

The UF polymer is dried overnight at 110 Celsius, allowed to cool, and sifted to remove clumps (using a standard baking sifter).

### Apparatus

1. Balance

2. 100 ml graduated cylinder

3. Powder funnel

### Procedure

1. Take a clean dry 100 ml graduated cylinder.

2. Charge about 40 g of powder by means of a powder funnel to the graduated cylinder.

3. Tap lightly the graduated cylinder on the bench top until all the air pockets along the sides of the cylinder have been filled in and until the level of powder does not change.

4. Read the volume of the powder in milliliters.

5. Weigh the cylinder in grams.

6. Report the Bulk Density in grams/ml.

$$Bulk Density = B - A$$

$$V$$

A=Empty cylinder weight

B=Cylinder weight plus sample

V=Volume of sample in cylinder in milliliters.

1. A method of treating hair and/or skin of a human or animal, the method comprising applying to the hair and/or skin a composition comprising particles of a urea formaldehyde polymer wherein the particles have an average size of less then 300 microns; an oil absorption value of greater than 40 g/100 g; and a bulk density of greater than 0.2 gcm⁻³.

2. A personal care composition comprising particles of a urea formaldehyde polymer having an average particle size of less than 300 microns; an oil absorption value of greater than 40 g/100 g; and a hulk density of greater than 0.2 gcm⁻³,

3. The personal care composition according to claim 2 which further comprises water and/or one or more organic solvents.

4. The personal care composition according to claim 2 which further comprises a surfactant.

5. The personal care composition according to claim 2 which further comprises an inorganic solid adjuvant salt.

6. The personal care composition according to claim 2 wherein the weight ratio of solid ingredients to liquid ingredients is from 5:1 to 1:2.

7. The personal care composition according to claim 2 which further comprises a fragrance.

8. The personal care composition according to claim 2 which further comprises a dye or colourant.

9. The personal care composition according to claim 2 which further comprises one or more ingredients selected from preservatives, dyes, fragrances, anti-caking agents, hair conditioning compounds, soft hold polymers, film-forming agents, thickeners, vitamins, proteins, UV filters, sunscreens, anti itch additives, anti fungal additives and anti lice additives,

10. The method according to claim 1 comprising applying to the hair and/or skin a composition as claimed in claim 2.

11. The method according to claim 1 wherein the hair is human hair.

12. The method according to claim 1 wherein the skin is human skin,

13. The personal care composition according to claim 2 which is in the form of a dry shampoo.

14. The method according to claim 11 which achieves one or more of the following effects: conditioning the hair, controlling static fly away, improving shine, removing oil from the hair, removing odours from the hair, improving dry controllability.

15. The personal care composition according to claim 2 comprising:
(a) from 10 to 90 wt % of the urea formaldehyde polymer;
(b) from 10 to 90 wt % of water, one or more organic solvents or a mixture thereof;
(c) 0 to 10 wt % of one or more surfactants; and
(d) 0 to 90 wt % of one or more inorganic solid adjuvant salts,

16. The method according to claim 10 wherein the hair is human hair.

17. The method according to claim 10 wherein the skin is human skin.

18. The method according to claim 16 which achieves one or more of the following effects; conditioning the hair, controlling static fly away, improving shine, removing oil from the hair, removing odours from the hair, improving dry controllability.
